(19) United States
(12) Patent Application Publication
(10) Pub. No.: US 2021/0065103 A1
(43) Pub. Date: Mar. 4, 2021

(54) SYSTEMS, DEVICES, AND METHODS FOR GENERATING PREPARATION, HANDLING, PACKAGING, AND DELIVERY INSTRUCTIONS AND VERIFYING INTEGRITY AND SAFETY IN NETWORK ORDER DELIVERY

(71) Applicant: Eduardo HAUSER, Hollywood, FL (US)

(72) Inventor: Eduardo HAUSER, Hollywood, FL (US)

(21) Appl. No.: 17/011,761

(22) Filed: Sep. 3, 2020

Related U.S. Application Data
(60) Provisional application No. 62/895,702, filed on Sep. 4, 2019.

(51) Int. Cl. G06Q 10/08; G06Q 30/06; G06Q 10/10; G06Q 10/06; G06Q 30/00; G16H 20/60; G16H 70/20; G06Q 50/26; G06Q 50/12; G06F 16/9535

(52) U.S. Cl. CPC G06Q 10/0832; G06Q 30/0633; G06Q 10/10; G06Q 10/06316; G06Q 30/0185; G06F 16/9535; G16H 70/20; G06Q 50/265; G06Q 50/12; G06Q 10/087; G16H 20/60

(57) ABSTRACT

Systems, devices, and methods for securing food integrity and safety during a delivery process. The method includes receiving an order placed by a consumer for food delivery from a provider. The order includes at least one item. The method also includes determining, from a provider profile, one or more ingredients contained in the at least one item. The method further includes retrieving, based on the one or more ingredients, guidelines comprising procedures for handling, packaging, and delivery of items containing the one or more ingredients. Additionally, the method includes generating instructions for packaging, handling, and delivery of the at least one item based on the guidelines. The method includes transmitting, over a network, an electronic copy of the instructions.

### PRIORITY

This application claims priority to U.S. Provisional Application No. 62/895,702 filed Sep. 4, 2019, the entire contents of which are incorporated by reference.

### FIELD

Embodiments hereof relate to systems, devices and methods for generating preparation, packing, handling, and delivery instructions and tracking integrity and safety in network order delivery.

### BACKGROUND

Restaurants and other food services and vendors have seen a dramatic rise in the demand for delivery of food items. This is due in part to the popularity of third-party food delivery services such as DOORDASH, UBER EATS, POSTMATES, GRUBHUB and others. Moreover, dining and shopping restrictions due to pandemics (e.g., stay-at-home orders, restrictions on in-person dining, shopping, etc.) have increased the demand of delivery of food items. This popularity, however, has given rise to safety and verification issues in the delivery process. When third-party food delivery services are used, the restaurants and other food services no longer have control of the food items once picked up by the delivery service (and the drivers lack the specialized handling training known my most restaurants and/or food services). Additionally, the restaurants and other food services generally only focus on packaging the food in containers that are on-hand with no thought give to controlling the food's conditions during delivery. Further, due to pandemics, food delivery verification, integrity, and safety have become important concerns due to possible spread of infections, diseases, viruses, etc.

These and other drawbacks exist with conventional network delivery services. These drawbacks are addressed by embodiments described herein.

### BRIEF SUMMARY

Embodiments of the present disclosure include systems, devices, and methods for generating preparation, packing, safe handling, and delivery instructions and tracking integrity and safety in network order delivery.

In one aspect, the present disclosure provides a method for securing food integrity and safety during a delivery process. The method includes receiving an order placed by a consumer for food delivery from a provider. The order includes at least one item. The method also includes determining, from a provider profile, one or more ingredients contained in the at least one item. The method further includes retrieving, based on the one or more ingredients, guidelines comprising procedures for handling, packaging, and delivery of items containing the one or more ingredients. Additionally, the method includes generating instructions for packaging, handling, and delivery of the at least one item based on the guidelines. The method includes transmitting, over a network, an electronic copy of the instructions.

In another aspect, a system includes a memory unit including software instructions and at least one processor configured to execute the software instructions to perform a method. The method includes receiving an order placed by a consumer for food delivery from a provider. The order includes at least one item. The method also includes determining, from a provider profile, one or more ingredients contained in the at least one item. The method further includes retrieving, based on the one or more ingredients, guidelines comprising procedures for handling, packaging, and delivery of items containing the one or more ingredients. Additionally, the method includes generating instructions for packaging, handling, and delivery of the at least one item based on the guidelines. The method includes transmitting, over a network, an electronic copy of the instructions.

In another aspect, a computer readable medium stores instructions that cause one or more processors to perform a method for securing food integrity and safety during a delivery process. The method includes receiving an order placed by a consumer for food delivery from a provider. The order includes at least one item. The method also includes determining, from a provider profile, one or more ingredients contained in the at least one item. The method further includes retrieving, based on the one or more ingredients, guidelines comprising procedures for handling, packaging, and delivery of items containing the one or more ingredients. Additionally, the method includes generating instructions for packaging, handling, and delivery of the at least one item based on the guidelines. The method includes transmitting, over a network, an electronic copy of the instructions.

### BRIEF DESCRIPTION OF DRAWINGS

The foregoing and other features and advantages of the present invention will be apparent from the following description of embodiments hereof as illustrated in the accompanying drawings. The accompanying drawings, which are incorporated herein and form a part of the specification, further serve to explain the principles of various embodiments described herein and to enable a person skilled in the pertinent art to make and use various embodiments described herein. The drawings are not to scale.

FIG. 1 illustrates a network delivery system, according to an embodiment disclosed herewith.

FIG. 2 illustrates the network delivery system of FIG. 1, according to an embodiment disclosed herewith.

FIG. 3 illustrates a process of generating instructions for preparation, handling, packaging, and delivery of items, according to an embodiment disclosed herewith.

FIG. 4 is a diagram illustrating operation of the network environment during the process of FIG. 3, according to an embodiment disclosed herewith.

FIGS. 5A-5C are diagrams illustrating example labels generated during the process of FIG. 3, according to an embodiment disclosed herewith.

FIG. 6 is a diagram illustrating examples of containers used during the process of FIG. 3, according to an embodiment disclosed herewith.

FIGS. 7A and 7B are diagrams illustrating another example of a label used during the process of FIG. 3, according to an embodiment disclosed herewith.

FIGS. 8A-8C are diagrams illustrating another example of a label used during the process of FIG. 3, according to an embodiment disclosed herewith.

FIGS. 9A-9C are diagrams illustrating examples of bag containers used during the process of FIG. 3, according to an embodiment disclosed herewith.

FIGS. 10A and 10B are diagrams illustrating another example of a bag container used during the process of FIG. 3, according to an embodiment disclosed herewith.

FIG. 11 illustrates a process of registering a provider, according to an embodiment disclosed herewith.

FIG. 12 is a diagram illustrating operation of the network environment during the process of FIG. 7, according to an embodiment disclosed herewith.

### DETAILED DESCRIPTION

Specific embodiments of the present invention are now described with reference to the figures. The following detailed description is merely exemplary in nature and is not intended to limit the present invention or the application and uses thereof. Furthermore, there is no intention to be bound by any expressed or implied theory presented in the preceding technical field, background, brief summary or the following detailed description.

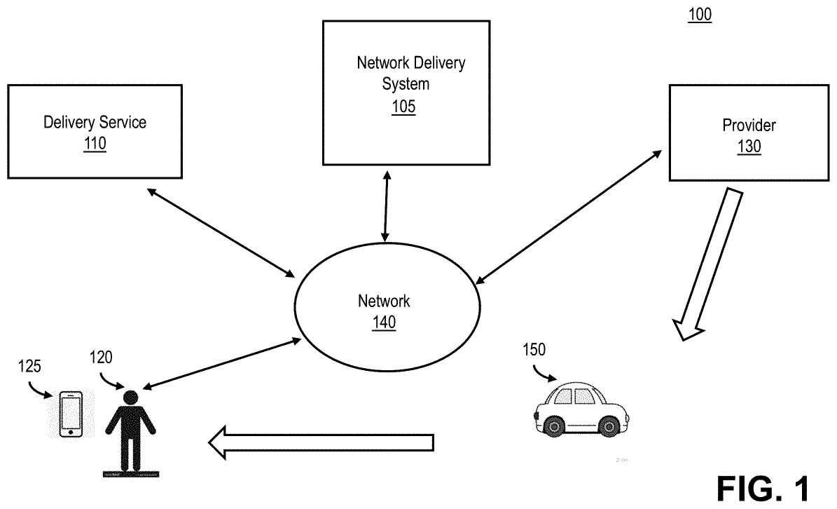

FIG. 1 illustrates a network delivery environment 100 for generating preparation, packing, handling, and delivery instructions and tracking integrity and safety in network order delivery in accordance with an embodiment hereof. One skilled in the art will realize that FIG. 1 illustrates one example of a network delivery environment and that existing components illustrated in FIG. 1 may be removed and/or additional components may be added to the network delivery environment 100 without departing from the scope of embodiments described herein.

As illustrated in FIG. 1, the network delivery environment 100 includes a network delivery system 105, a delivery service 110, a consumer 120, and a provider 130 communicating over a network 140. The consumer 120 can place an order with the delivery service 110 (or directly with the provider 130) with an electronic device 125 over a network 140. The order can include one or more menu items offered by the provider 130 to be delivered by a delivery agent 150. As described herein, an order identifies menu items selected by the consumer 120 and optionally can also identify preferences of the consumer 120. A preference can include allergies of the consumer 120 (e.g., peanut allergy, dairy allergy, etc.), dietary restrictions/preferences (e.g., lactose intolerance, vegan, vegetarian, kosher, diabetic, kosher, etc.), custom food preparation instructions, custom packaging instructions, custom delivery instructions, etc. A menu item can include any offering from the provider 130. For example, a menu item can include prepared food dishes (e.g., turkey sandwich, ceviche, etc.), unprepared food items (e.g., a piece of fruit, a piece of raw meat, a bag of chips, etc.), drinks, condiments, non-food items, or any other item offer by a provider 130.

As described herein, the delivery service 110 can include any entity facilitating delivery of the menu item from the provider 130 to the consumer 120. The delivery service 110 can operate to receive the order from the consumer 120, forward the order to the provider 130, schedule and direct the physical delivery by the delivery agent 150, etc. In an embodiment, as illustrated in FIG. 1, the delivery service 110 can be a third-party service that operates separate from the provider 130, such as DOORDASH, UBER EATS, POSTMATES, GRUBHUB, etc. In another embodiment, the delivery service 110 can be part of or directly controlled by the provider 130 such as a delivery service operated by the provider 130.

As described herein, the consumer 120 can include any entity, e.g., person, computer system, software, etc. placing an order with a delivery service 110. For example, the consumer 120 can be a person placing an order with a local restaurant and/or food service using a mobile application associated with the delivery service 110, installed on the electronic device 125. As described herein, the provider 130 can include any entity providing menu items for order or request by the consumer 120. For example, the provider 130 can include restaurants, cafes, food trucks, ghost kitchens, catering services, grocery stores, convenience stores, etc.

As described herein, the delivery agent 150 can include any entity that physically delivers the menu items to the consumer 120. In an embodiment, the delivery agent 150 can be an employee of the delivery service 110 and/or the provider 130. In another embodiment, the delivery agent 150 can be a contractor or agent of the delivery service 110 and/or provider 130.

In an embodiment, the system 105 operates in coordination with the delivery service 110, the provider 130, and/or the consumer 120 to provide instructions for packaging, handling, preparation, and delivery orders for the provider 130 and/or delivery agent 150 and to provide handing instructions and verification to the consumer 120. As described below in further detail, the system 105 maintains a database of guidelines that specify procedure for packaging, handling, preparing, and delivering menu items based on a number of factors such as the ingredients contained in menu items, allergens, dietary restrictions, caloric values, government regulations regarding the safe handling of food, industry standards/practices regarding the safe preparation/handling of food, food science standards and procedures, and any other predetermined or custom factors associated with proper and safe handling of food.

In operation, the system 105 receives the details of an order placed by the consumer 120 and analyzes the order to determine one or more instructions for the preparation, handling, packaging, and delivery of the menu items contained in the order. For each order analyzed, the system 105 determines one or more decision parameters that guide the identification of the guidelines and the generation of the instructions for the preparation, handling, packaging, and delivery of the menu items. For example, the decision parameters can include information such as the ingredients contained in the menu items, preparation of the menu items, presence of allergens, intended use, target user group, preferences of the consumer 120, relevant information about the provider 130, and any other relevant information. Based on the decision parameters, the system 105 searches, identifies, and extracts relevant guidelines from the database and generates instructions based on the relevant guidelines. Once identified, the system 105 provides, over the network 140 to the delivery service 110 and/or the provider 130, electronic copies of the instructions along with electronic copies of one or more labels that can be affixed to containers holding the menu items. The label can contain any information that may be relevant to the handling and packaging by the provider 130, relevant to handling and delivery by the delivery agent 150, and relevant to the verification, handling, consumption, preparation, and storage by the consumer 120.

In an embodiment, the system 105 provides analysis of orders and generation of instructions and labels as a service which may be subscribed to and/or accessed by the delivery service 110 and/or the provider 130. For example, the system 105 can provide remote access to the functionality of the system 105 through requests and responses transmitted over the network 140. In another example, the system 105 can provide software applications and/or modules that are executed by the delivery service 110 and/or provider 130 to access the analysis of orders and generation of instructions and labels whether locally at the delivery service 110 and/or the provider 130, or remotely across the network 140. In another embodiment, one or more function performed by the system 105 can be provided directly to the consumer 120.

The system 105 can be configured as a server (e.g., having one or more server blades, processors, etc.), a personal computer (e.g., a desktop computer, a laptop computer, etc.), a smartphone, a tablet computing device, and/or other device that can be programmed to generate and provide instructions for preparing, handling, packaging, and delivering menu items. In an embodiment, any or all of the functionality of the system 105 can be performed as part of a cloud computing platform. In an embodiment, any or all of the functionality of the system 105 can be performed by or integrated in another system, such as the systems of the delivery service 110 and/or the systems of the provider 130.

The electronic device 125 can include any type of electronic device and/or computer system capable of communicating with any of the system 105, the delivery service 110, the provider 130, and the delivery agent 150 over the network 140. For example, the electronic devices can include a personal computer (e.g., a desktop computer, a laptop computer, etc.), a smartphone, a tablet computing device, a smartwatch, video game controller or console, etc. The electronic device 125 can include any hardware (e.g., processors, storage, I/O devices, etc.) and software to place orders for menu items over the network 140. In an embodiment, any of the computing functionality of the electronic device 125 can be performed as part of a cloud computing platform.

The network 140 can include any type of communication channel or network for transmitting data between the system 105, the delivery service 110, the provider 130, and the delivery agent 150. In an embodiment, the network 140 can include one or more communication networks such as local area networks and wide area network, e.g., the Internet. In another embodiment, the network 140 can include inter-device communication channels such as data buses, e.g., serial, wireless communication channels, e.g., WIFI, Bluetooth, and the like.

While FIG. 1 illustrates one system 105, one delivery service 110, one consumer 120, one provider 130, one network 140, and one delivery agent 150, the network delivery environment 100 can include any number of systems 105, delivery services 110, consumers 120, providers 130, networks 140, and delivery agents 150, and the network delivery environment 100 is not limited to including one of each. Embodiments described herein having specific numbers of systems 105, delivery services 110, consumers 120, providers 130, networks 140, and delivery agents 150 are provided for illustrative purposes only and are not intended to be limiting.

While FIG. 1 illustrates the system 105, the delivery service 110, the provider 130 as being separate systems, one skilled in the art will realize that any of the system 105, the delivery service 110, the provider 130 may be embodied in a single system and/or device. For example, the delivery service 110 and/or the provider 130 can incorporate the systems and functionality of the system 105.

Storage devices, as discussed herein, include non-transitory computer readable media, and any media or computer memory that stores data and/or instructions that cause a machine to operate in a specific fashion. Such non-transitory media may include non-volatile media and/or volatile media. Non-volatile media includes, for example, optical or magnetic disks, as well as various forms of solid-state media. Volatile media includes dynamic memory. Common forms of non-transitory media include, for example, a floppy disk, a flexible disk, hard disk, solid state drive, magnetic tape, or any other magnetic data storage medium, a CD-ROM, DVD, Blu-Ray, any other optical data storage medium, any physical medium with patterns of holes, a RAM, a PROM, and EPROM, a FLASH-EPROM, NVRAM, any other memory chip or cartridge, and networked versions of the same.

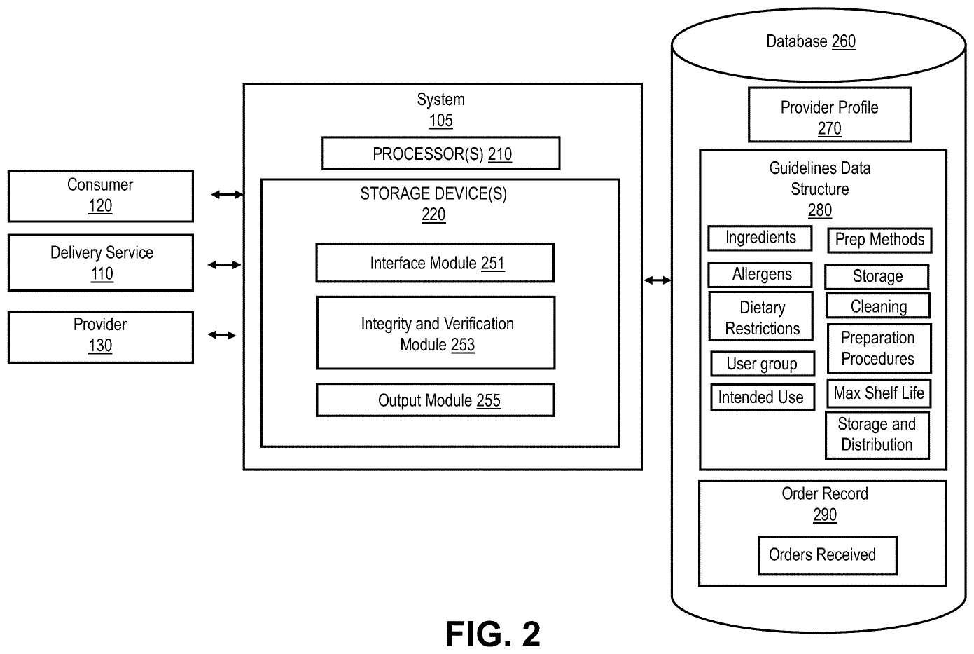

FIG. 2 is a diagram illustrating the system 105 in accordance with an embodiment hereof. One skilled in the art will realize that FIG. 2 illustrates one example of the hardware and software components included therein and that existing components illustrated in FIG. 2 may be removed and/or additional components may be added to the system 105.

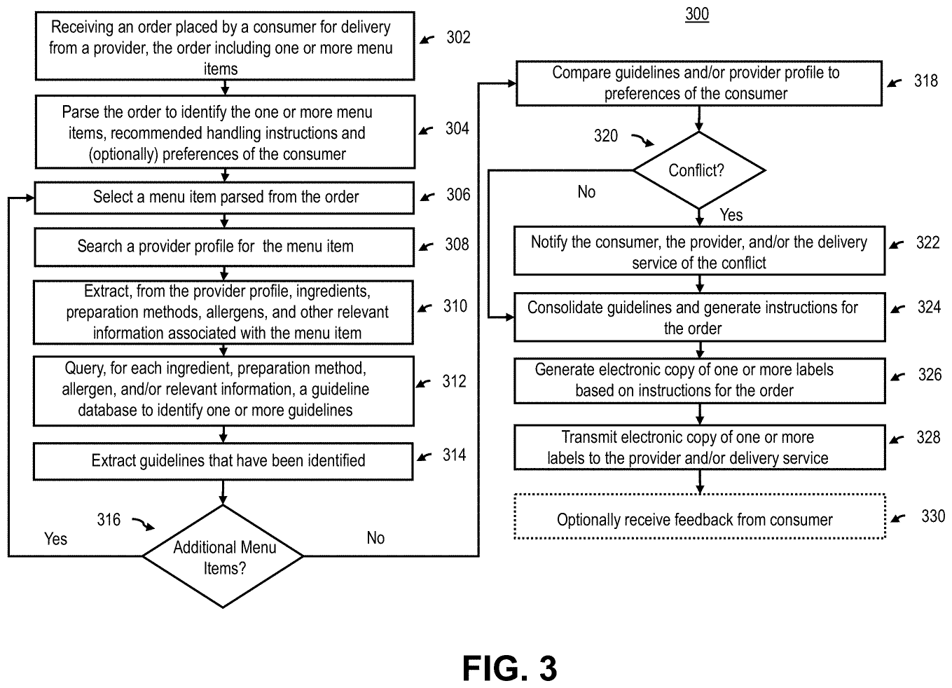

As illustrated in FIG. 2, the system 105 includes one or more processors 210 (also interchangeably referred to herein as processors 210, processor(s) 210, or processor 210 for convenience), one or more storage device(s) 220, and/or other components. The processor 210 is programmed by one or more computer program instructions stored on the storage device 220. For example, the processor 210 is programmed by an interface module 251, an integrity module 253, a verification module 255, and an output module 257. Additionally, the system 105 includes and/or communicates with a database 260. As used herein, for convenience, the various modules and systems will be described as performing an operation, when, in an embodiment, the modules and systems program the processor 210 (and therefore the system 105) to perform the operation. For example, the operations and functionality of the modules and system can be embodied in software that is executed by the processor 210. In another embodiment, the operations and functionality of the modules and system can be embodied in software, hardware, and combinations thereof. Further details and features of the system 105 configured for implementing features of the described invention may be understood with respect to FIGS. 3-8.

The interface module 251 is configured to interface and communicate with the delivery service 110, the provider 130, and/or the consumer 120 (or any other component, device or system of the network delivery environment 100) in order to receive details of an order placed by the consumer 120. For example, the interface module 251 can include a user interface in which a user and/or user device can provide an order. Likewise, for example, the interface module 251 can include an application programming interface (“API”) that can be called by other modules, software, application, and devices to invoke the operations of the system 105. Additionally, for example, the interface module 251 can include a communication interface for communicating with the delivery service 110 (or any other component, device or system of the network delivery environment 100) over the network 140 or other communication channel using any suitable transmission protocol including, e.g., HTTP, TCP, UDP, etc. Also, for example, the interface module 251 can include a database and/or storage interface for retrieving orders from a storage system, storage device, and/or data structure maintained by delivery service 110, the provider 130, and/or the consumer 120 (or any other component, device or system of the network delivery environment 100).

The integrity and verification module 253 is configured to generate handling, preparation, and/or delivery instructions (hereinafter “instructions”) for the menu items contained in the order. The integrity and verification module 253 can communicate with a database 260. The database 260 stores a provider profile 270 and a guideline data structure 280. The integrity and verification module 253 can be configured to parse an order placed by the consumer 120 and extract any relevant information required to generate the instructions.

The provider profile 270 stores any relevant information on the provider 130. The information can include a listing of the menu items offered by the provider, the ingredients contained in the menu items, the preparation methods of the menu items, or any other relevant information for the menu items. The provider profile 270 can also store other information about the provider 130 such as allergens used in the preparation of menu items that may cause cross-contamination.

The guideline data structure 280 stores various data tables that outline guidelines for handling, preparing, packaging, delivering, and storing menu items. The data tables can include tables that identify ingredients by type, preparation method by type, allergens and potential severity levels, dietary restrictions, storage and distribution methods by type, packaging methods, delivery methods, cleaning methods, preparation procedures, user groups, intended use, maximum shelf life, etc. The data tables in the guideline data structure 280 can include data keys and/or cross-referencing in order for various ingredients to be associated with caloric values, allergens, dietary restrictions, preparation method, storage methods, packaging methods, delivery methods, and cleaning methods, and allow the integrity and verification module 253 to search and extract guidelines based on the any data contained in the guideline data structure 280.

The integrity and verification module 253 can also be configured to generate one or more labels to be placed on containers that hold the menu items. The integrity and verification module 253 can generate the labels based on the instructions that were generated. The labels are configured to be placed on the containers holding the menu items or other items that are being delivered to the consumer 120. The labels provide a visual indication to the consumer of the integrity of the delivery of the menu items. The labels can provide information such as the ingredients contained in the menu items, possible allergens, the preparation methods used in preparing the menu items, the containers that should be used and details of the containers, suggested delivery times, intended use, maximum shelf life, storage/reheating instructions for the consumer, caloric values of food items, suggested disposal and recycling instructions for the containers, etc.

The database 260 can also include an order record 290. The order record 290 can include a record of orders that have been handled by the system 105. For each order, the order record 290 can include any information relevant to the order handled by the system 105 such as identification of the order, provider, and delivery service; items in the order; details of the order; results of the analysis by the system 105 (e.g., allergy alerts, preparation time, packaging instructions, delivery instructions, reheat instructions, etc.) and the like.

Once the handling, preparation, and/or delivery instructions are generated, the instructions are passed to the output module 255. The output module 255 is configured to output the instructions. In an embodiment, the output module 255 can be configured to provide or transmit the instructions to the delivery service 110 and/or the provider 130 (or any other component, device or system of the network delivery environment 100). For example, the output module 255 may include an API that can be called by other modules, software, application, and devices to provide or transmit the instructions. Additionally, for example, the output module 255 may include a network interface to provide or transmit the instructions over the network 140. Likewise, for example, the output module 255 may include a communication interface, e.g., serial bus, Bluetooth, WIFI, NFC, or any other communication technology, for communicating (wired or wirelessly) to provide or transmit the instructions. In an embodiment, the output module 255 can be configured to store the instructions in storage, e.g., the database 260. For example, the output module 225 may output the instructions and the labels to electronic devices, such as a display, a speaker, a mobile phone, a tablet computer, a printer, etc.

While the system 105 is described above as including the interface module 251, the integrity and verification module 253, and the output module 255, one skilled in the art will realize the functionality of these modules can be implemented in any number of software modules or application, whether executed by the system 105 or other computer system. Likewise, one skilled in the art will realize that the functionality of any of the interface module 251, the integrity and verification module 253, and the output module 255 can be implemented in hardware or combinations of hardware and software.

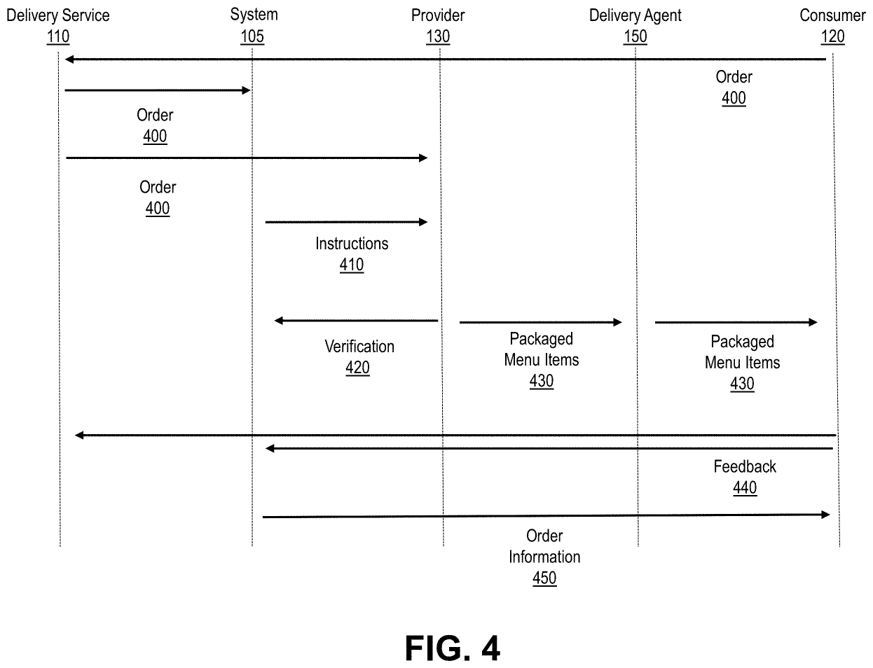

FIGS. 3 and 4 are a flow chart and diagram, respectively, illustrating process 300 for generating handling, preparation, packaging, and delivery instructions, in accordance with an embodiment hereof. The process 300 is performed on a computer system having one or more physical processors programmed with computer program instructions that, when executed by the one or more physical processors, cause the computer system to perform the method. The one or more physical processors are referred to below as simply the processor. In embodiments, the process 300 is carried out via the system 105 as described herein. The system 105 represents an example of a hardware and software combination configured to carry out the process 300, but implementations of the process 300 are not limited to hardware and software combination of the system 105. Additional details regarding each of the operations of the process may be understood according to the description of the network delivery environment, as described above.

In an operation 302, the process 300 includes receiving an order placed by a consumer for delivery from a provider. The order includes one or more menu items offered by the provider. The order can also include preferences of the consumer.

For example, as illustrated in FIG. 4, the consumer 120, using the electronic device 125, can place an order with the delivery service 110. Once received, the delivery service 110 can provide the order to the system 105. While FIG. 4 illustrates the delivery service 110 provides the order to the system 105, the consumer 120 and/or the provider 130 can provide the order to the system 105. For example, if the provider 130 is providing delivery services, the provider 130 can provide the order to the system 105. Likewise, for example, if the system 105 is operating as the delivery service, the consumer 120 can provide the order directly to the system 105.

In an operation 304, the process 300 includes parsing the order to identify the one or more menu items, recommended handling instructions, and (optionally) preferences of the consumer. For example, the system 105 can search the order and identify the provider 130 that is the subject of the order, the menu items selected in the order, and any preferences included in the order.

In an operation 306, the process 300 includes selecting a menu item parsed from the order. For example, the system 105 can iteratively review each menu item in order to generate instructions.

In an operation 308, the process 300 includes searching a provider profile for the menu item. For example, the system 105 can identify the provider profile 270 contained in the database 260 and retrieve the provider profile 270 from the database 260.

In an operation 310, the process 300 includes extracting, from the provider profile, ingredients, preparation methods, allergens, and other relevant information associated with the menu item. For example, the system 105 can identify the menu items in the provider profile 270 and extract ingredients, preparation methods, allergens, and other relevant information indexed with the menu item.

In an operation 312, the process 300 includes querying, for each ingredient, preparation method, allergen, dietary guidelines, handling guidelines, packaging guidelines, delivery guidelines, and other relevant information, a guideline database or data structures to identify one or more guidelines. In an operation 314, the process 300 includes extracting the one or more guidelines that have been identified.

For example, the system 105 can search the guidelines data structure 280 for each ingredient, preparation method, allergen, and other relevant information extracted from the provider profile 270. The system 105 can extract any guidelines that are indexed to the search items, e.g., handling guidelines, storage guidelines, packaging guidelines, preparation guidelines, and/or delivery guidelines.

In an operation 316, the process 300 includes determining if additional menu items are contained in the order. If additional menu items are contained, the process 300 returns to operation 306. If no additional menu items are contained, the process proceeds to operation 318. In operation 318, the process includes comparing the one or more guidelines and/or provider profile to preferences of the consumer. Based on the comparison, the process 300 includes determining if a conflict exists. If a conflict exists, in an operation 322, the process 300 includes notifying the consumer, the provider, and/or the delivery service of the conflict.

For example, the system 105 can provide a description of the conflict to the consumer, the provider, and/or the delivery service of the conflict. Likewise, for example, the system 105 can provide suggestion for mitigating a conflict, e.g., suggest a modification to the menu item, suggest packaging, canceling the order, modifying the order, determine and suggest a different provider that can avoid the conflict, determine and select items without conflicts, etc.

If no conflict exists, in an operation 324, the process 300 includes consolidating the one or more guidelines and generating instructions for the order. For example, for each menu item in the order the system 105 can examine each of the guidelines extracted and can make a decision for preparation, handling, packaging, and/or delivery instructions for each menu item. For example, for a menu item, the system 105 can iteratively examine the extracted guidelines for each ingredient and can make a decision as to preparation, handling, packaging, and/or delivery instructions based the extracted guidelines and predetermined rules. For instance, if a menu item had ingredient 1 that recommends storage in container A and ingredient 2 that recommend storage in container B, the system 105 can select either container A or container B based on the rules.

In embodiments, the rules can be based on the priority of the guidelines in the guidelines data structure 280. For example, the rules can prioritize guidelines that are directed to health and safety such as allergies and safe food handling, storage, and delivery. Likewise, the rules can utilize the severity of an outcome if a guideline is not followed in determining which guidelines to utilize. For example, FDA usually groups these hazards in two categories: illness and injury. In other words, the FDA guidelines are intended to eliminate the risk of injuries and illnesses arising from food items.

Additionally, for example, after instructions are generated for each menu item, the system 105 can perform a global review of the guidelines and consolidate the instructions for the entire order. For instance, in the complete order, the system 105 can determine that container A is recommended for menu item 1 and that container B is recommended for menu item 2. In this example, the system 105 can examine menu item 1 and menu item 2 and determine that container C can address the guidelines for both container A and container B.

In an operation 326, the process 300 includes generating an electronic copy of one or more labels based on the instructions for the order. The labels are configured to be placed on the containers holding the menu items and may include features to prevent the items from tampering. The labels may provide a visual indication to the consumer of the integrity of the delivery of the menu items. In some embodiments, the labels can provide information such as the ingredients contained in the menu items, the preparation methods used in preparing the menu items, dietary compliance, the containers that should be used and details of the containers, suggested delivery times, suggested storage/reheating instructions for the consumer, nutritional information, etc.

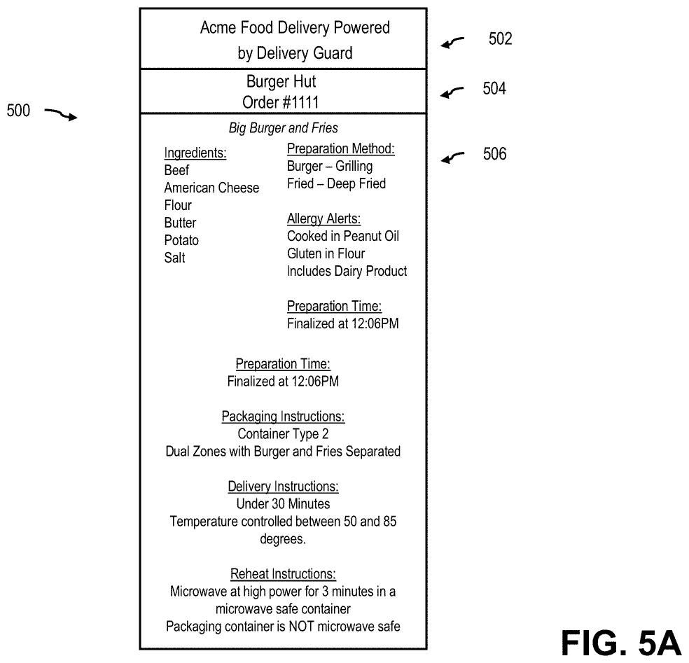

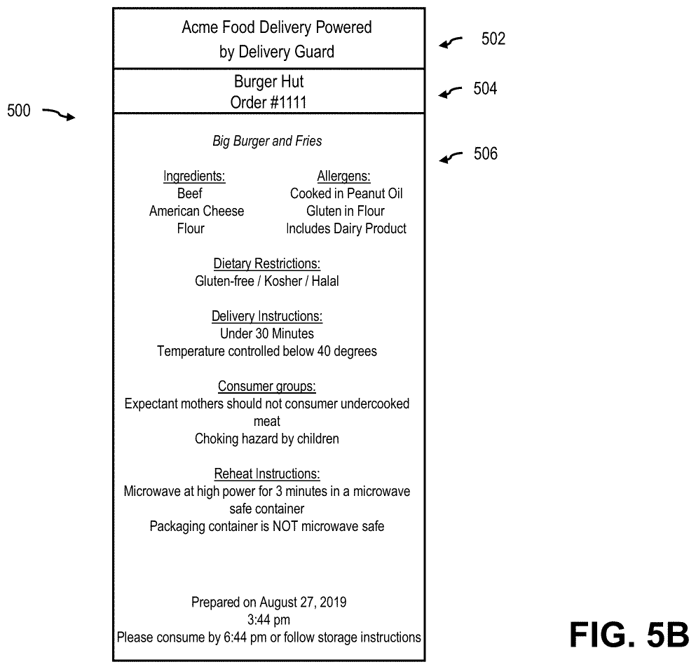

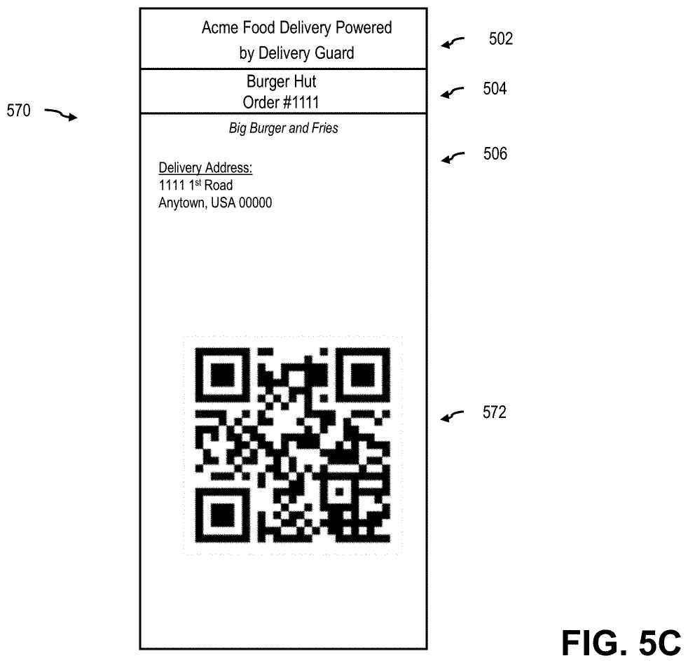

FIG. 5A illustrates one example of a label 500 that can be generated by the system 105. As illustrated in FIG. 5A, the label 500 includes a field 502 that identifies the delivery service 110 and that the services of the system 105 were utilized. The label 500 also includes a field 504 that identifies the provider 130 and the order that is the subject of the label. The label 500 also includes a field 506 that identifies the details of the order, the results of the analysis by the system 105, such as allergy alerts, preparation time, packaging instructions, delivery instructions, and reheat instructions. FIG. 5B illustrates another example of a label 550 that can be generated by the system 105.

In some embodiments, the label generated by the system 105 can include human readable and/or computer readable information (e.g., network address, electronic links, etc.) that allows a consumer 120 to remotely access information such as details of the order and the results of the analysis by the system 105, such as allergy alerts, preparation time, packaging instructions, delivery instructions, and reheat instructions. For example, the label can include network addresses, barcodes, Quick Response (QR) codes, etc. that allow the consumer 120 (or other entity) to access information about order via the network 140 using an electronic device such as the electronic device 125. FIG. 5C illustrates another label 570 that can be generated by the system 105. As illustrated in FIG. 5C, the label 570 includes a field 502 that identifies the delivery service 110 and that the services of the system 105 were utilized. The label 570 also includes a field 504 that identifies the provider 130 and the order that is the subject of the label. The label 570 also includes a field 571 that identifies the delivery information, e.g., address of the consumer 120. The label 570 also includes a computer readable identifier 572, e.g., QR code, that encodes information about the order. For example, the computer readable identifier 572 can encode a locator, identifier, or tracker that points to a network location where the information about an order is stored, e.g., the order record 290 in the database 260. In this example, the consumer 120 can read the computer readable identifier 572 using the electronic device 125 and be directed to information about the order such as the details of the order and the results of the analysis by the system 105, such as allergy alerts, preparation time, packaging instructions, delivery instructions, and reheat instructions.

In an operation 328, the process 300 includes transmitting an electronic copy of the one or more labels and the instructions to the provider and/or delivery service. For example, as illustrated in FIG. 4, the system 105 can provide electronic copies the instructions and labels 410 to the provider 130 over the network 140. In some embodiments, the details of the order can be stored by the system 105, for example, in the order record 290. After the instructions and labels 410 are received by the provider 130, the provider 130 fulfills (preparing, handling, packaging) the order 400, produces physical copies of the labels, and places the labels on the containers holding the menu items to produce packaged menu items 430. The provider 130 can also send a verification 420 (e.g., verification message, images of the packaged menu item, etc.) to the system 105 that the instructions were followed, and the labels were affixed to the packaged menu items 430. The delivery agent 150 then delivers the packaged menu item 430 to the consumer 120.

In an optional operation 330, the process includes receiving feedback from the consumer. In some embodiments, the feedback 440 can include a request for information about the order. For example, if the label includes a human and/or computer readable identifier, the system 105 can receive a network request from the consumer 120 using the electronic device 125. In response, the system 105 can transmit or send order information 450 about the order, e.g., details of the order and the results of the analysis by the system 105, such as allergy alerts, preparation time, packaging instructions, delivery instructions, and reheat instructions. In some embodiments, the feedback 440 can include a consumer feedback about the delivery and the items received. For example, the feedback 440 by the consumer 120 can include details on the packaging, delivery, and condition of the menu items such as whether or not the delivery was concluded on time, if safe handling instructions were followed, the conditions the menu/food items were received in, among others. The feedback 440 by the consumer 120 can include a rating of the delivery agent 150, the provider 130, and/or the delivery service that can be utilized by the system 105, the delivery service 110, and/or the provider 130 in future operations. The feedback by the consumer 120 can include images of the packaged menu items that can be utilized by the system 105, the delivery service 110, and/or the provider 130 that can be used for verification and future improvement.

The above describes an illustrative flow of an example process 300 for generating handling, preparation, packaging, and delivery instructions. The process as illustrated in FIG. 3 is exemplary only, and variations exist without departing from the scope of the embodiments disclosed herein. The steps may be performed in a different order than that described, additional steps may be performed, and/or fewer steps may be performed.

In the process 300 described above, the provider profile may store the menu item associated with the ingredients, preparation method, etc. Then, in operations 306-316, for an order, the process can iteratively determine guidelines for each menu item by accessing the provider profile to identify ingredients, preparation method, etc. and search the guideline database. In another embodiment, for each menu item offered by a provider 130, the system 105 can identify guidelines and pre-generate instructions for each menu item (and possible modifications) at the time the provider 130 registers with the system 105. In this embodiment, the process 300 can be altered to identify and extract the pre-generated instructions based on the menu items identified in the order. Additionally, in the example, if an order contains a menu item (or a modification to a menu item) that does not pre-generated instructions, the process can perform the operations 306-316.

In the process 300 described above, the provider 130 can package the items in conventional container for delivery to the consumer 120. FIG. 6 illustrate various examples of containers that can be selected by the system 105 and utilized by the provider 130 in packaging. As illustrated in FIG. 6, the containers can include a resealable container, for example, a clamshell container 610. While 610 is illustrated as a clamshell container, one skilled in the art will realize that the resealable container can be any type of container that can be opened to placed one or more food items and closed to seal the one or more food items, for example, a bag, a box and sealable lid or top, a pouch with a sealable opening, etc. Likewise, the containers can include container that can hold the food items and/or other container, for example, a bag 620. In any embodiment, the container can be construed of any material to hold and transport food items and/or other container, for example, paper, plastic, fabric, cardboard, metal, etc. Additionally, in any embodiment, the container can be constructed of and/or include insulating materials to maintain a temperate of the one or more food items contained therein.

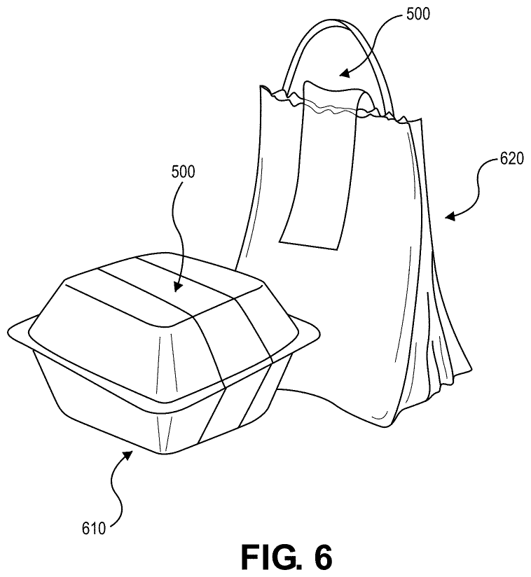

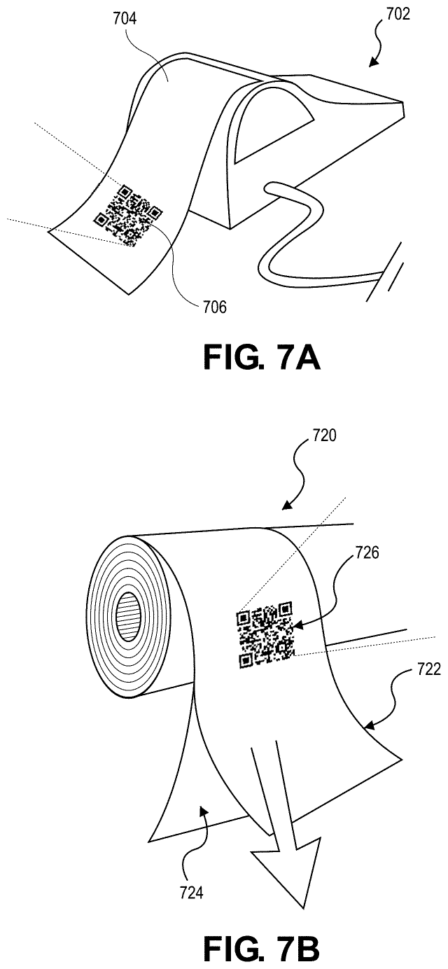

In the process 300, the labels produced by the provider 130 can be physically created using any type of printing process. In some embodiments as illustrated in FIG. 7A, the provider 130 can include a printer 702 that is coupled to one or more computer systems. The printer 702 can be configured to print information about an order on one or more labels 704. For example, the printer 702 can print the label 704 that includes a computer readable identifier 706 that encoded with a locator, identifier, or tracker, as discussed above. In some embodiments, the printer 702 can be a conventional printer that is owned and operated by the provider 130. In some embodiments, the printer 702 can be a propriety printer provided by the system 105.

In some embodiments, the provider 130 can utilize preprinted labels supplied by the system 105. For example, as illustrated in FIG. 7B, the system 105 can provide a roll 720 of preprinted labels 722. Prior to providing the preprinted labels 722 to the provider 130, the preprinted labels 722 can generated to include a computer readable identifier 726. At the time of generation, the computer readable identifier 726 can be encoded with a locator, identifier, or tracker that is not associated with a particular network location or address. Once an order is placed, the computer readable identifier 726 can be associated with locator, identifier, or tracker that points to a network location where the information about an order is stored, e.g., the order record 290 in the database 260.

In any embodiment, the labels utilized by the provider 130 can include one or more features that allow a consumer 120 to determine if the items being delivered have undergone tampering. For example, as illustrated in FIG. 7B, the label 720 can include a removable backing 724 that covers an adhesive on a back side of the label 720. While not illustrated, the label 704 illustrated in FIG. 7A can include a removable backing and adhesive similar to label 720. As illustrated in FIG. 6, once a label has been generated, the label 720 can be affixed to a container in a manner such that tampering with the container can be recognized by a consumer 120. For example, a container 730 may be a clamshell type container and a container 732 can be a bag type container. The label 720 can be positioned on the container 730 and/or container 732 such that opening the container 730 and/or container 732 is sealed by the label 720. That is, the label 720 can be adhered to both sides of the container 730 and/or container 732 such that the label 720 spans the opening of the container 730 and/or container 732. If anyone attempts to open the container 730, e.g., the delivery agent 150, the label 720 and/or the container 730/732 will be damaged.

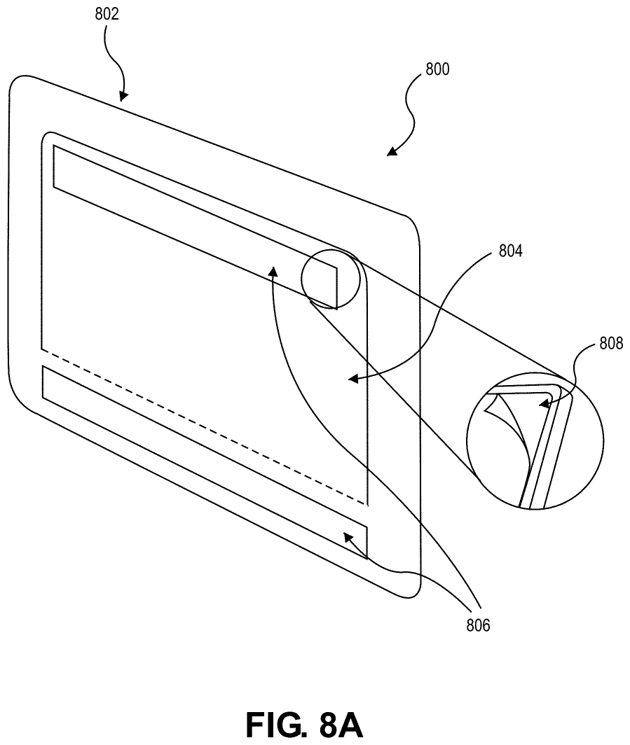

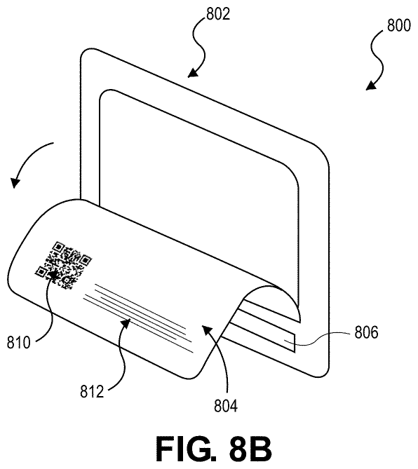

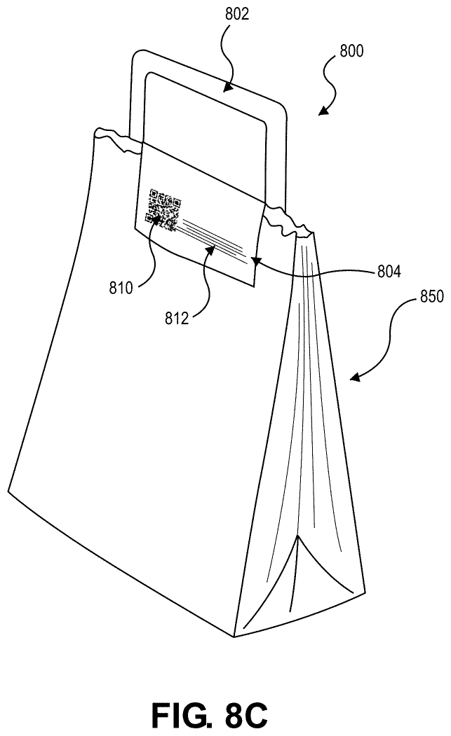

FIGS. 8A-8C illustrate another example of a label 800 that can be utilized with the process 300. As illustrated in FIG. 8A, the label 800 includes a body 802 and a foldable tab 804 located in a center portion of the body. The foldable tab 804 is configured to be moved or folded relative to the body 802, as illustrated in FIG. 8B. The body 802 and the foldable tab 804 include a removable strip 806 that covers an adhesive 808. As illustrated in FIG. 8B, one side of the foldable tab 804 can operate to convey the information about the order such as a computer readable identifier 810 and order information 812, as described above in reference to FIGS. 5A-5C.

The label 800 operates as a tamper indicator and a handle for a container. As illustrated in FIG. 8C, once a label has been printed and/or associated with order information, the label 800 can be affixed to a container 850 in a manner such that tampering with the container 850 can be recognized by a consumer 120. For example, a container 850 can be a bag type container. The label 800 can be positioned on the container 850 such that opening of the container 850 is sealed by the label 800. That is, the foldable tab 804 of the label 800 can be folded relative to the body 802. Once folded, the foldable tab 804 can be adhered to one side of the container 850 and the body 802 can be adhered to another side of the container 850 such that the label 800 spans the opening of the container 850. If anyone attempts to open the container 850, e.g., the delivery agent 150, the label 800 and/or the container 850 will be damaged. Additionally, once folded and adhered, the body 802 operates as a handle for the container 850.

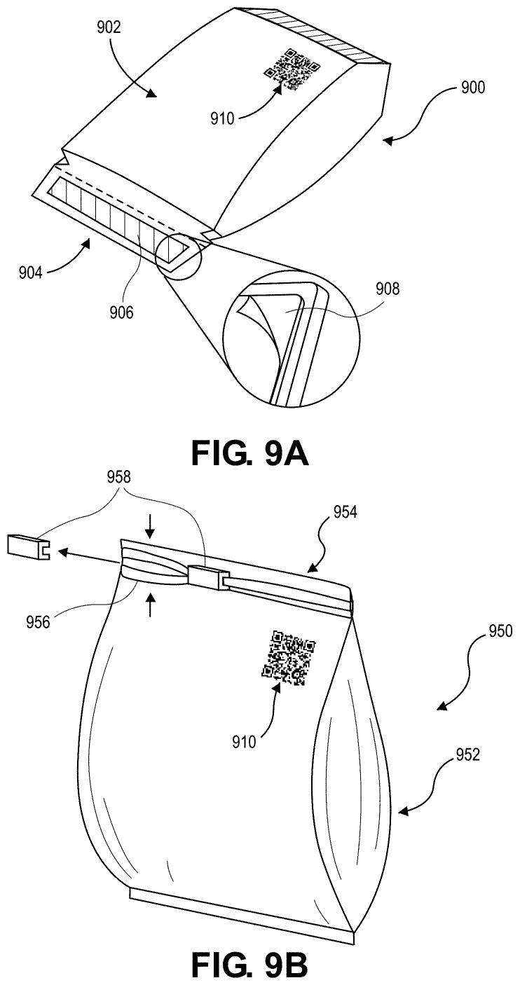

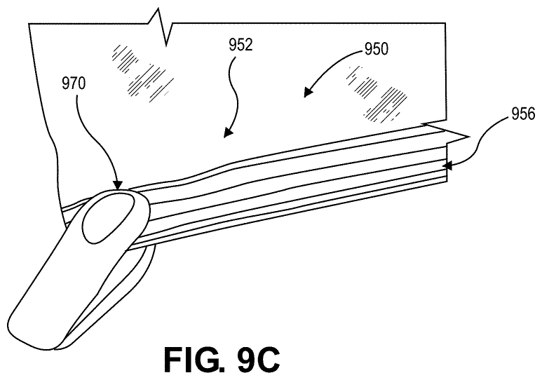

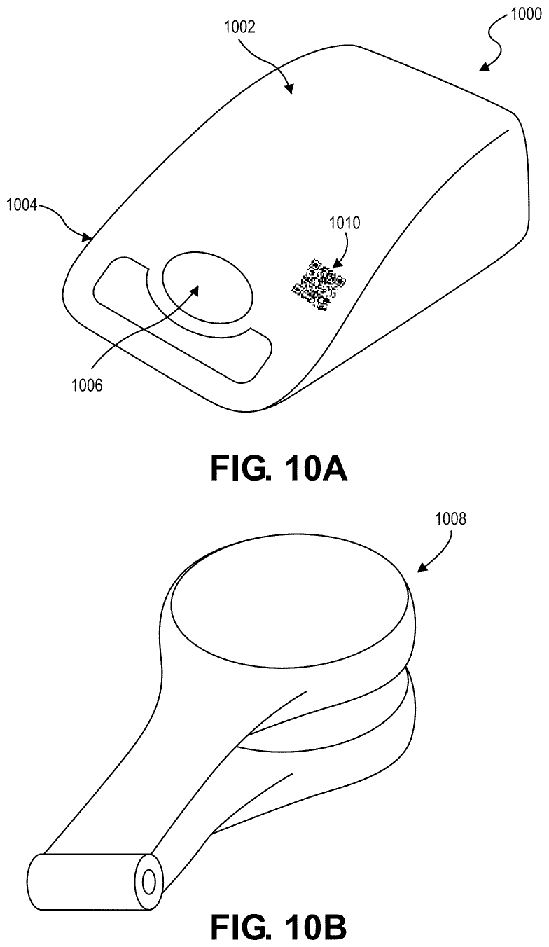

In some embodiments, the system 105 may provide customized containers for delivering items to the consumer 120. FIGS. 9A-9C, 10A, and 10B illustrate examples of container that can be utilized by the provider 130. FIG. 9A illustrates one example of a bag container 900. As illustrated in FIG. 9A, the bag container 900 includes a body 902 and a foldable tab 904. The body 902 defines a cavity in which item associated with an order can be placed. The foldable tab 904 is configured to be moved or folded relative to the body 902 in order to seal the cavity defined by the body 902. The foldable tab 904 includes a removable strip 906 that covers an adhesive 908. That is, the foldable tab 904 can be folded relative to the body 902. Once folded, the foldable tab 904 can be adhered to one side of the body 902 such that the opening of the body 904 is sealed. If anyone attempts to open the container 900, e.g., the delivery agent 150, the container 900 will be damaged.

In some embodiments, the body 902 of the container 900 can include information about the order associated with the container 900. For example, as illustrated, the body 902 can include a computer readable identifier 910. At the time of generation, the computer readable identifier 910 can be encoded with a locator, identifier, or tracker that is not associated with a particular network location or address. Once an order is placed, the computer readable identifier 910 can be associated with locator, identifier, or tracker that points to a network location where the information about an order is stored, e.g., the order record 290 in the database 260. One skilled in the art will realize that additionally labels, such as label described herein, can be affixed to the container 900.

FIG. 9B illustrates another example of a bag container 950. As illustrated in FIG. 9B, the bag container 950 includes a body 952 and a sealable tab 954. The body 952 defines a cavity in which item associated with an order can be placed. The sealable tab 954 includes a zipper strip 956 with a removable zipper 958. To seal the body 952, the zipper 958 can be moved along the zipper strip 956 in order to seal the opening of the body 952. Once sealed, the zipper 958 can be removed from the zipper strip 956 thereby sealing the body 952 such that opening the bag container 950 damages the bag container 950. If anyone attempts to open the container 950, e.g., the delivery agent 150, the container 950 will be damaged. In some embodiments, the body 952 of the container 950 can include information about the order associated with the container 950. For example, as illustrated, the body 952 can include a computer readable identifier 910. At the time of generation, the computer readable identifier 910 can be encoded with a locator, identifier, or tracker that is not associated with a particular network location or address. Once an order is placed, the computer readable identifier 910 can be associated with locator, identifier, or tracker that points to a network location where the information about an order is stored, e.g., the order record 290 in the database 260. One skilled in the art will realize that additionally labels, such as label described herein, can be affixed to the container 950.

In some embodiments, the zipper 958 of the zipper strip 956 can be omitted. In this embodiment, as illustrated in FIG. 9C, a zipper tool 970 can be utilized to seal the zipper strip 956. The zipper tool 970 operators to join the components of the zipper strip 956 thereby sealing the body 952 such that opening the bag container 950 damages the bag container 950. If anyone attempts to open the container 950, e.g., the delivery agent 150, the container 950 will be damaged.

FIGS. 10A and 10B illustrate another example of a bag container 1000. As illustrated in FIG. 10A, the bag container 1000 includes a body 1002, a handle 1004, and a sealable feature 1006. The body 1002 defines a cavity in which item associated with an order can be placed. The sealable feature 1006 operators to seal an opening of the body 1002 such that a consumer 120 can identify tampering. In some embodiment, the sealable feature 1006 can include an adhesive that is inert at ambient temperatures and becomes active when heated. To active to the adhesive, a heat press (or iron) 1008 can be utilized to heat the sealable feature 1006 and active the adhesive. That is, a user can close the heat press 1008 one either side of the sealable feature 1006. Once closed, the heat press 1008 can apply thermal energy to the sealable feature 1006 thereby heating the adhesive and sealing the body 1002. If anyone attempts to open the container 100, e.g., the delivery agent 150, the container 100 will be damaged. In some embodiments, the body 1002 of the container 1000 can include information about the order associated with the container 1000. For example, as illustrated, the body 1002 can include a computer readable identifier 1010. At the time of generation, the computer readable identifier 1010 can be encoded with a locator, identifier, or tracker that is not associated with a particular network location or address. Once an order is placed, the computer readable identifier 1010 can be associated with locator, identifier, or tracker that points to a network location where the information about an order is stored, e.g., the order record 290 in the database 260. One skilled in the art will realize that additionally labels, such as label described herein, can be affixed to the container 1000.

In any of the embodiments described above, the containers can be constructed to different sizes, shapes, and dimensions in order to accommodate items of varying sizes or multiple items.

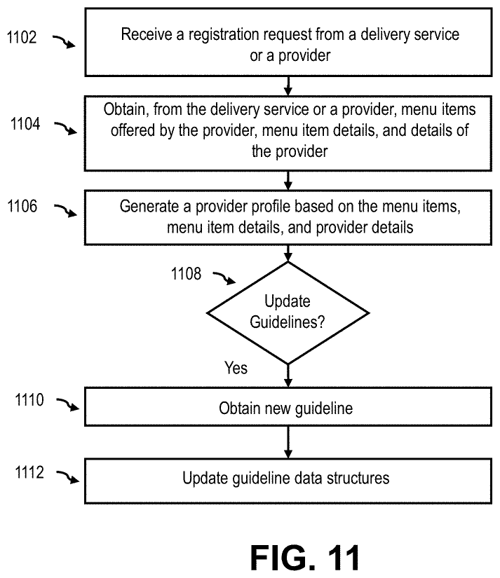

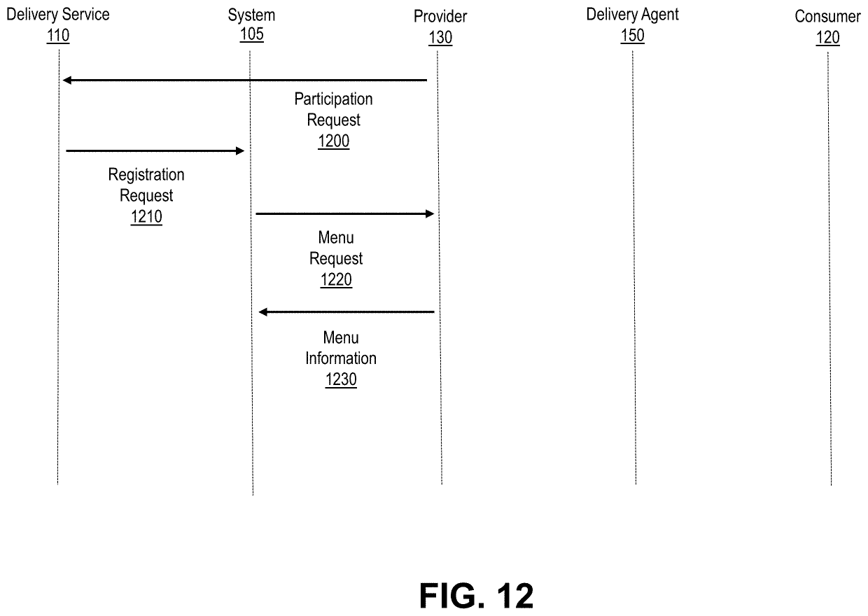

FIGS. 11 and 12 are a flow chart and diagram, respectively, illustrating process 1100 for generating handling, preparation, packaging, and delivery instructions, in accordance with an embodiment hereof. The process 1100 is performed on a computer system having one or more physical processors programmed with computer program instructions that, when executed by the one or more physical processors, cause the computer system to perform the method. The one or more physical processors are referred to below as simply the processor. In embodiments, the process 1100 is carried out via the system 105 as described herein. The system 105 represents an example of a hardware and software combination configured to carry out the process 1100, but implementations of the process 1100 are not limited to hardware and software combination of the system 105. Additional details regarding each of the operations of the process may be understood according to the description of the network delivery environment, as described above.

In an operation 1102, the process 1100 includes receiving a registration request from a delivery service or a provider. In an embodiment, the delivery service 110 can provide the registration request to the system 105. For example, as illustrated in FIG. 8, the provider 130 can provide a participation request 1200 to the delivery service 110. The participation request 1200 can identify an intent by the provider 130 to offer menu items through the delivery service. The participation request 1200 can also include details about the menu items offered by the provider 130, e.g., identification and cost of the menu items, the ingredients contained in the menu items, the preparation method for the menu items, potential allergens in the menu items, caloric value of the menu items, potential conflicts with dietary restrictions, containers used to store menu item, etc. The participation request 1200 can also include general details about the provider such as cleaning methods used in the restaurant and/or food service, possible allergen present but not in the menu items, location, etc. Once received, the delivery service 110 can provide a registration request 1210 to the system 105. The registration request 1210 can include a request to create a provider profile for the provider 130 and the information to populate the provider profile that was received from the provider 130. In an embodiment, the system 105 can received the registration request 1210 and/or the information from the participation request directly from the provider 130.

In an operation 1104, the process 1100 includes obtaining, from the delivery service or a provider menu items, offered by the provider, menu item details, and details of the provider. In an embodiment, the delivery service 110 can provide the details of the provider 130 to the system 105. In another embodiment, the provider 130 can provide the details directly to the system 105. For example, as illustrated in FIG. 8, the system 105 can transmit a menu request 1220 to the provider 130. In response, the provider 130 can transmit menu information 1230 to the system 105. The menu information 1230 can include an identification and cost of the menu items, the ingredients contained in the menu items, the preparation method for the menu items, potential allergens in the menu items, containers used to store menu item, etc. In another embodiment described above, operation 1104 may be optional when the provider 130 provides the menu information in the registration request.

In an operation 1106, the process 1100 includes generating a provider profile based on the menu items, menu item details, and provider details. For example, the system 105 can generate a new provider profile 270 with the received information and store the new provider profile 270 in the database 260.

In an operation 1108, the process 1100 includes determining if the guidelines data structure should be updated. If not, the process 1100 can end. If yes, in an operation 712, the process 1100 includes updating the guideline data structures. For example, a menu item from the new provider 130 may contain an unknown item (e.g., an ingredient, preparation method, etc.) that does not have an associated guideline in the guideline data structure 280. The system 105 can examine various food safety and handling sources (e.g., government regulation, food science sources, industry standards, etc.) and determine one or more new guidelines. The system 105 can update and/or generate data tables in the guideline data structure 280 with the one or more new guidelines.

Further embodiments of the present disclosure are described below.

Embodiment 1 is a method for securing food integrity and safety during a delivery process. The method can include receiving an order placed by a consumer for food delivery from a provider, the order comprising at least one item. The method can include determining, from a provider profile, one or more ingredients contained in the at least one item. The method can include retrieving, based on the one or more ingredients, guidelines comprising procedures for handling, packaging, and delivery of items containing the one or more ingredients. The method can include generating instructions for packaging, handling, and delivery of the at least one item based on the guidelines. The method can include transmitting, over a network, an electronic copy of the instructions.

Embodiment 2 is the method of Embodiment 1 wherein the method further includes generating at least one label based on the instructions. The at least one label includes one or more of instructions for packaging the at least one item, details of the order, details of a preparation of the at least one item, instructions for delivering the at least one item, allergens contained in the at least one item, instruction for reheating the at least one item, and instructions for storing the at least one item.

Embodiment 3 is the method of Embodiment 1 wherein the method further includes generating at least one label based on the instructions. The at least one label includes a computer readable identifier that provides access to one or more of instructions for packaging the at least one item, details of the order, details of a preparation of the at least one item, instructions for delivering the at least one item, allergens contained in the at least one item, instruction for reheating the at least one item, and instructions for storing the at least one item.

Embodiment 4 is the method of Embodiment 1, wherein the instructions for packaging, handling, and delivery include one or more of: steps for packaging the at least one item according the guidelines, an identification of at least one predefined container for transporting the at least one item, and instructions for transporting the at least one item. The guidelines include one or more of: guidelines for allergens, guidelines for meeting dietary restrictions and preferences, guidelines for handling predetermined ingredients according to health regulations, guidelines for packaging food items according to the health regulations, best known practices and the predetermined ingredients, guidelines for storing the food items according to the health regulations, best known practices, guidelines for consuming the food items according to the health regulations, best known practices, guidelines for transporting the food items according to the health regulations, best known practices, and guidelines for selecting predefined containers based on the predetermined ingredients.

Embodiment 5 is the method of Embodiment 1, wherein the method further includes determining, from a provider profile, a preparation method associated with the at least one item. The instructions are generated based on the preparation method.

Embodiment 6 is the method of Embodiment 5, wherein the order includes at least one consumer preference. The method further includes comparing the at least one consumer preference to at least one of the one or more ingredients and the preparation method. The method includes, when the at least one consumer preference conflicts with the one or more ingredients or the preparation method, transmitting a notification of the conflict, wherein the notification is transmitted, over the network, to one or more of the consumers, a delivery service, and provider. The at least one consumer preference comprises a food allergy.

Embodiment 7 is the method of Embodiment 1, wherein the method further includes prior to receiving the order, receiving a request to register the provider. The request to register is received from at least one of the providers and a delivery service. The method includes transmitting, over the network, a request for menu information comprising items offered by the provider and predetermined ingredients contained in the items. The method includes generating the provider profile based on the menu information.

Embodiment 8 is the method of Embodiment 1, wherein the method further includes receiving, from the provider prior to delivery, a handling verification that verifies handling and packaging of the at least one item according to the instructions. The method includes receiving, from the consumer after delivery, feedback on a condition of the at least one item. The method includes verifying compliance with the instructions based on a comparison of the handling verification and the feedback. The method includes notifying one or more of the consumers, a delivery service, and the provider of the verification.

Embodiment 9 is a system for securing food integrity and safety during a delivery process. The system includes a memory unit including software instructions. The system includes at least one processor configured to execute the software instructions to perform a method. The method includes receiving an order placed by a consumer for food delivery from a provider, the order including at least one item. The method includes determining, from a provider profile, one or more ingredients contained in the at least one item. The method includes retrieving, based on the one or more ingredients, guidelines comprising procedures for handling, packaging, and delivery of items containing the one or more ingredients. The method includes generating instructions for packaging, handling, and delivery of the at least one item based on the guidelines. The method includes transmitting, over a network, an electronic copy of the instructions.

Embodiment 10 is the system of Embodiment 9, wherein the instructions for packaging, handling, and delivery include one or more of: steps for packaging the at least one item according the guidelines, an identification of at least one predefined container for transporting the at least one item, and instructions for transporting the at least one item. The guidelines include one or more of: guidelines for allergens, guidelines for meeting dietary restrictions and preferences, guidelines for handling predetermined ingredients according to health regulations, guidelines for packaging food items according to the health regulations, best known practices and the predetermined ingredients, guidelines for storing the food items according to the health regulations, best known practices, guidelines for consuming the food items according to the health regulations, best known practices, guidelines for transporting the food items according to the health regulations, best known practices, and guidelines for selecting predefined containers based on the predetermined ingredients.

Embodiment 11 is the system of Embodiment 9, wherein the method further includes determining, from a provider profile, a preparation method associated with the at least one item. The instructions are generated based on the preparation method.

Embodiment 12 is the system of Embodiment 11, wherein the order includes at least one consumer preference. The method further includes comparing the at least one consumer preference to at least one of the one or more ingredients and the preparation method. The method includes, when the at least one consumer preference conflicts with the one or more ingredients or the preparation method, transmitting a notification of the conflict. The notification is transmitted, over the network, to one or more of the consumers, a delivery service, and provider.

Embodiment 13 is the system of Embodiment 9, wherein the method further includes prior to receiving the order, receiving a request to register the provider. The request to register is received from at least one of the provider and a delivery service. The method includes transmitting, over the network, a request for menu information comprising items offered by the provider and predetermined ingredients contained in the items. The method includes generating the provider profile based on the menu information.

Embodiment 14 is the system of Embodiment 9, wherein the method further includes generating at least one label based on the instructions. The at least one label comprises one or more of instructions for packaging the at least one item, details of the order, details of a preparation of the at least one item, instructions for delivering the at least one item, allergens contained in the at least one item, instruction for reheating the at least one item, and instructions for storing the at least one item.

Embodiment 15 is a computer readable medium storing instructions that cause one or more processors to perform a method for securing food integrity and safety during a delivery process. The method includes receiving an order placed by a consumer for food delivery from a provider, the order comprising at least one item. The method includes determining, from a provider profile, one or more ingredients contained in the at least one item. The method includes retrieving, based on the one or more ingredients, guidelines comprising procedures for handling, packaging, and delivery of items containing the one or more ingredients. The method includes generating instructions for packaging, handling, and delivery of the at least one item based on the guidelines. The method includes generating at least one label based on the instructions. The method includes transmitting, over a network, an electronic copy of the instructions.

Embodiment 16 is the computer readable medium of Embodiment 15, wherein the method further includes generating at least one label based on the instructions. The at least one label includes one or more of instructions for packaging the at least one item, details of the order, details of a preparation of the at least one item, instructions for delivering the at least one item, allergens contained in the at least one item, instruction for reheating the at least one item, and instructions for storing the at least one item.

Embodiment 17 is the computer readable medium of Embodiment 15, wherein the instructions for packaging, handling, and delivery includes one or more of: steps for packaging the at least one item according the guidelines, an identification of at least one predefined container for transporting the at least one item, and instructions for transporting the at least one item. The guidelines include one or more of: guidelines for allergens, guidelines for meeting dietary restrictions and preferences, guidelines for handling predetermined ingredients according to health regulations, guidelines for packaging food items according to the health regulations, best known practices and the predetermined ingredients, guidelines for storing the food items according to the health regulations, best known practices, guidelines for consuming the food items according to the health regulations, best known practices, guidelines for transporting the food items according to the health regulations, best known practices, and guidelines for selecting predefined containers based on the predetermined ingredients.

Embodiment 18 is the computer readable medium of Embodiment 15, wherein the method further includes determining, from a provider profile, a preparation method associated with the at least one item. The instructions are generated based on the preparation method.

Embodiment 19 is the computer readable medium of Embodiment 18, wherein the order includes at least one consumer preference. The method further includes comparing the at least one consumer preference to at least one of the one or more ingredients and the preparation method. The method includes, when the at least one consumer preference conflicts with the one or more ingredients or the preparation method, transmitting a notification of the conflict. The notification is transmitted, over the network, to one or more of the consumers, a delivery service, and provider.

Embodiment 20 is the computer readable medium of Embodiment 15, wherein the method further includes prior to receiving the order, receiving a request to register the provider. The request to register is received from at least one of the provider and a delivery service. The method includes transmitting, over the network, a request for menu information comprising items offered by the provider and predetermined ingredients contained in the items. The method includes generating the provider profile based on the menu information.

While various embodiments according to the present disclosure have been described above, it should be understood that they have been presented by way of illustration and example only, and not limitation. It will be apparent to persons skilled in the relevant art that various changes in form and detail can be made therein without departing from the spirit and scope of the present disclosure. Thus, the breadth and scope of the present disclosure should not be limited by any of the above-described exemplary embodiments but should be defined only in accordance with the appended claims and their equivalents. It will also be understood that each feature of each embodiment discussed herein, and of each reference cited herein, can be used in combination with the features of any other embodiment. Stated another way, aspects of the above methods may be used in any combination with other methods described herein or the methods can be used separately. All patents and publications discussed herein are incorporated by reference herein in their entirety.

What is claimed is:

1. A method for securing food integrity and safety during a delivery process, the method comprising:
receiving an order placed by a consumer for food delivery from a provider, the order comprising at least one item;
determining, from a provider profile, one or more ingredients contained in the at least one item;
retrieving, based on the one or more ingredients, guidelines comprising procedures for handling, packaging, and delivery of items containing the one or more ingredients;
generating instructions for packaging, handling, and delivery of the at least one item based on the guidelines; and
transmitting, over a network, an electronic copy of the instructions.

2. The method of claim 1, the method further comprising:
generating at least one label based on the instructions, wherein the at least one label comprises one or more of instructions for packaging the at least one item, details of the order, details of a preparation of the at least one item, instructions for delivering the at least one item, allergens contained in the at least one item, instruction for reheating the at least one item, and instructions for storing the at least one item.

3. The method of claim 1, the method further comprising:
generating at least one label based on the instructions, wherein the at least one label comprises a computer readable identifier that provides access to one or more of instructions for packaging the at least one item, details of the order, details of a preparation of the at least one item, instructions for delivering the at least one item, allergens contained in the at least one item, instruction for reheating the at least one item, and instructions for storing the at least one item.

4. The method of claim 1, wherein the instructions for packaging, handling, and delivery comprise one or more of:
steps for packaging the at least one item according the guidelines,
an identification of at least one predefined container for transporting the at least one item, and
instructions for transporting the at least one item; and
wherein the guidelines comprise one or more of:
guidelines for allergens,
guidelines for meeting dietary restrictions and preferences,
guidelines for handling predetermined ingredients according to health regulations,
guidelines for packaging food items according to the health regulations, best known practices and the predetermined ingredients,
guidelines for storing the food items according to the health regulations, best known practices,
guidelines for consuming the food items according to the health regulations, best known practices,
guidelines for transporting the food items according to the health regulations, best known practices, and
guidelines for selecting predefined containers based on the predetermined ingredients.

5. The method of claim 1, the method further comprising:
determining, from a provider profile, a preparation method associated with the at least one item, wherein the instructions are generated based on the preparation method.

6. The method of claim 5, wherein the order comprises at least one consumer preference, and the method further comprises:
comparing the at least one consumer preference to at least one of the one or more ingredients and the preparation method; and
when the at least one consumer preference conflicts with the one or more ingredients or the preparation method, transmitting a notification of the conflict, wherein the notification is transmitted, over the network, to one or more of the consumers, a delivery service, and provider, wherein the at least one consumer preference comprises a food allergy.

7. The method of claim 1, the method further comprising:
prior to receiving the order, receiving a request to register the provider, wherein the request to register is received from at least one of the providers and a delivery service;
transmitting, over the network, a request for menu information comprising items offered by the provider and predetermined ingredients contained in the items; and
generating the provider profile based on the menu information.

8. The method of claim 1, the method further comprising:
receiving, from the provider prior to delivery, a handling verification that verifies handling and packaging of the at least one item according to the instructions;
receiving, from the consumer after delivery, feedback on a condition of the at least one item;
verifying compliance with the instructions based on a comparison of the handling verification and the feedback; and
notifying one or more of the consumers, a delivery service, and the provider of the verification.

9. A system for securing food integrity and safety during a delivery process, the system comprising:
a memory unit including software instructions; and
at least one processor configured to execute the software instructions to perform a method comprising:
receiving an order placed by a consumer for food delivery from a provider, the order comprising at least one item;
determining, from a provider profile, one or more ingredients contained in the at least one item;
retrieving, based on the one or more ingredients, guidelines comprising procedures for handling, packaging, and delivery of items containing the one or more ingredients;
generating instructions for packaging, handling, and delivery of the at least one item based on the guidelines; and
transmitting, over a network, an electronic copy of the instructions.

10. The system of claim 9, wherein the instructions for packaging, handling, and delivery comprise one or more of:
steps for packaging the at least one item according the guidelines,
an identification of at least one predefined container for transporting the at least one item, and
instructions for transporting the at least one item; and
wherein the guidelines comprise one or more of:
guidelines for allergens,
guidelines for meeting dietary restrictions and preferences,
guidelines for handling predetermined ingredients according to health regulations,
guidelines for packaging food items according to the health regulations, best known practices and the predetermined ingredients,
guidelines for storing the food items according to the health regulations, best known practices,
guidelines for consuming the food items according to the health regulations, best known practices,
guidelines for transporting the food items according to the health regulations, best known practices, and
guidelines for selecting predefined containers based on the predetermined ingredients.

11. The system of claim 9, the method further comprising:
determining, from a provider profile, a preparation method associated with the at least one item, wherein the instructions are generated based on the preparation method.

12. The system of claim 11, wherein the order comprises at least one consumer preference, and the method further comprises:
comparing the at least one consumer preference to at least one of the one or more ingredients and the preparation method; and
when the at least one consumer preference conflicts with the one or more ingredients or the preparation method, transmitting a notification of the conflict, wherein the notification is transmitted, over the network, to one or more of the consumers, a delivery service, and provider.

13. The system of claim 9, the method further comprising:
prior to receiving the order, receiving a request to register the provider, wherein the request to register is received from at least one of the provider and a delivery service;
transmitting, over the network, a request for menu information comprising items offered by the provider and predetermined ingredients contained in the items; and
generating the provider profile based on the menu information.

14. The system of claim 9, the method further comprising:
generating at least one label based on the instructions, wherein the at least one label comprises one or more of instructions for packaging the at least one item, details of the order, details of a preparation of the at least one item, instructions for delivering the at least one item, allergens contained in the at least one item, instruction for reheating the at least one item, and instructions for storing the at least one item.

15. A computer readable medium storing instructions that cause one or more processors to perform a method for securing food integrity and safety during a delivery process, the method comprising:
receiving an order placed by a consumer for food delivery from a provider, the order comprising at least one item;
determining, from a provider profile, one or more ingredients contained in the at least one item;
retrieving, based on the one or more ingredients, guidelines comprising procedures for handling, packaging, and delivery of items containing the one or more ingredients;
generating instructions for packaging, handling, and delivery of the at least one item based on the guidelines;
generating at least one label based on the instructions; and
transmitting, over a network, an electronic copy of the instructions.

16. The computer readable medium of claim 15, the method further comprising:
generating at least one label based on the instructions, wherein the at least one label comprises one or more of instructions for packaging the at least one item, details of the order, details of a preparation of the at least one item, instructions for delivering the at least one item, allergens contained in the at least one item, instruction for reheating the at least one item, and instructions for storing the at least one item.

17. The computer readable medium of claim 15, wherein the instructions for packaging, handling, and delivery comprise one or more of:
steps for packaging the at least one item according the guidelines,
an identification of at least one predefined container for transporting the at least one item, and
instructions for transporting the at least one item; and
wherein the guidelines comprise one or more of:
guidelines for allergens,
guidelines for meeting dietary restrictions and preferences,
guidelines for handling predetermined ingredients according to health regulations,
guidelines for packaging food items according to the health regulations, best known practices and the predetermined ingredients,
guidelines for storing the food items according to the health regulations, best known practices,
guidelines for consuming the food items according to the health regulations, best known practices,
guidelines for transporting the food items according to the health regulations, best known practices, and
guidelines for selecting predefined containers based on the predetermined ingredients.

18. The computer readable medium of claim 15, the method further comprising:
determining, from a provider profile, a preparation method associated with the at least one item, wherein the instructions are generated based on the preparation method.

19. The computer readable medium of claim 18, wherein the order comprises at least one consumer preference, and the method further comprises:
comparing the at least one consumer preference to at least one of the one or more ingredients and the preparation method; and
when the at least one consumer preference conflicts with the one or more ingredients or the preparation method, transmitting a notification of the conflict, wherein the notification is transmitted, over the network, to one or more of the consumers, a delivery service, and provider.

20. The computer readable medium of claim 15, the method further comprising:
prior to receiving the order, receiving a request to register the provider, wherein the request to register is received from at least one of the provider and a delivery service;
transmitting, over the network, a request for menu information comprising items offered by the provider and predetermined ingredients contained in the items; and
generating the provider profile based on the menu information.
